(19) United States
(12) Patent Application Publication
(10) Pub. No.: US 2014/0273003 A1
(43) Pub. Date: Sep. 18, 2014

(54) SUPER-RESOLUTION FLUORESCENCE LOCALIZATION MICROSCOPY

(71) Applicant: New York University, New York, NY (US)

(72) Inventors: Eli Rothenberg, New York, NY (US); David Fenyo, New York, NY (US); Dylan Reid, Rancho Cucamonga, CA (US); Mario Delmar, New York, NY (US); Marina Cerrone, New York, NY (US); Esperanza Agullo-Pascual, New York, NY (US)

(73) Assignee: New York University, New York, NY (US)

(21) Appl. No.: 14/209,704

(22) Filed: Mar. 13, 2014

Related U.S. Application Data
(60) Provisional application No. 61/782,973, filed on Mar. 14, 2013.

(51) Int. Cl. G01N 33/50; G06F 19/12

(52) U.S. Cl. CPC G01N 33/5091; G01N 33/502; G06F 19/12
USPC 435/7.1; 702/19

(57) ABSTRACT

The present invention relates generally ultrasensitive assays for use in diagnostics and in methods of drug screening and personalizing therapy for an individual patient. Specifically, the present invention relates to improved imaging and computational methods for detecting molecular phenotypes.

### CROSS REFERENCE TO RELATED APPLICATIONS

This application claims priority to U.S. Provisional Application No. 61/782,973 filed Mar. 14, 2013, reference which is hereby made in entirety.

### STATEMENT OF GOVERNMENT-SPONSORED RESEARCH

This invention was made with United States government support awarded by the following agencies: National Institutes of Health, National Heart, Lung, and Blood Institute (No. R01-HL106632) and National Institute of General Medical Science (No. R01-GM57691). The United States government has certain rights in the invention.

### FIELD OF THE INVENTION

The present invention relates generally to ultrasensitive assays for use in diagnostics and in methods of drug screening and personalizing therapy for an individual patient. Specifically, the present invention relates to improved methods of detecting molecular phenotypes.

### BACKGROUND OF THE INVENTION

The advent of high-throughput sequencing has opened up a new chapter in the study of genetics by allowing deep sequencing of whole populations of organisms and cells, as well as enhanced diagnostic capabilities of individuals. However, exploiting genome-wide sequence data for functional studies is challenging, as it is often not easy to discern functionally relevant genomic variation at the molecular level from changes without phenotypic effects. Further, even in individuals who are genetically predisposed to develop certain disorders, phenotypic variation at the molecular level will often determine the time and severity of onset, as well as the efficacy of a variety of treatments.

For example, familial cardiomyopathies result from mutations in genes that code for proteins that form the cardiac structures. In some cases, the disruption in the structure is not evident, even at the traditional microscopic level, until the individual reaches adulthood. Importantly, these patients often have life-threatening arrhythmias before even knowing that they have a cardiac disease. Even though the disease runs in the family, not every member will be affected. It is critical to discern which family member is at risk of developing the disease and also, which family member is at risk of sudden death before the structural disease becomes manifest. Risk stratification in inherited cardiac diseases is a crucial component of the medical approach to the affected families.

Cardiac structures are formed by the proper aggregation of molecules into confined subdomains (regional proteomes). Distancing between molecules can prevent their proper integration and as such, their ability to form a structure with the appropriate rigidity, strength and flexibility. Similarly, the electrical event that precedes the cardiac contraction results from the coordinated effort of macromolecular complexes. In the same manner as a building may show its weakening because of the formation of cracks along the wall, small separations between elements of a proteome are to be the first event in the progression of a life-threatening cardiomyopathy or inherited arrhythmia disease such as Brigade syndrome, where a structural defect often is not evident at the light microscopy level.

Light microscopy is often used as a diagnostic tool for cardiac structural disease. However, recognition of an abnormality in cell structure is limited by the power of optical resolution (250 nm). This resolution allows detection of changes in the micro-scale, such as variations in the size of the cell or its organelles, loss of specific proteins that can be fluorescently labeled or drastic disruption in their localization. However, this level of resolution fails to detect the very early events, namely, the loss of integrity of the proteomic complex in which the proteins reside.

Molecular phenotypes are also implicated in the mutation and deregulation of genes which are key players in complex DNA damage response pathways and left unchecked can lead to cancers. These proteins are modulators of recruitment and signaling which mediate proper DNA repair. The ability of cells to repair damage to the genome is essential for cell viability and function. DNA damage response is also linked to decreased treatment efficacy through increased resistance to radio and chemotherapeutic agents.

For example, genes regulating expression of human epidermal growth factor receptor type 2, which is a key biomarker that is overexpressed in a wide variety of carcinomas including breast, ovarian, prostate, and lung cancer. Since HER2 overexpression plays an important role in aggressive tumor behavior and poor clinical outcome, the early-stage detection and quantification of HER2 is clinically relevant and could be used for selection of optimal therapy for individual patients. However, common diagnostic methods rely on fluorescence in situ hybridization (FISH) and/or immunohistochemistry (IHC). These methods, although commonly used in clinical practice, have several limitations. Most notably, they require tissue removal from the body, which restricts their analysis only to the sampled parts and may not properly represent the overall tumor characteristics. The variability in scoring between these techniques, whether as a result of true heterogeneity or artifacts in preparation, has led to decreased reliability of the final HER2 status determination.

Current methods of cancer tissue cytopathology and cytogenetics rely on optical, fluorescence, and confocal microscopy. These methods are relatively crude, however, and their capability to resolve and localize molecular targets in the tissue is limited.

Still further, detection of genetic transformation and protein localization in autologous cells during drug screening processes is an essential step as we move towards truly personalized medicine. However, presently utilized methods of determining binding affinities and mapping molecular changes and characteristics that arise from drug treatment are often stymied by insufficiently sensitive detection methods.

There remains, therefore, a need for more sensitive techniques for use in diagnostic assays and methods that are dependent on molecular phenotypes, and in methods of drug screening and personalizing therapy for an individual patient.

### SUMMARY OF THE INVENTION

The present invention relates to imaging and computational assays developed to address the problems with existing techniques outlined above.

Additional features, advantages, and embodiments of the present disclosure may be set forth from consideration of the following detailed description, drawings, and claims. Moreover, it is to be understood that both the foregoing summary of the present disclosure and the following detailed description are exemplary and intended to provide further explanation without further limiting the scope of the present disclosure claimed.

In one aspect, the present invention provides a method of determining the prognosis for an individual with a genetic predisposition to familial cardiomyopathy, said method comprising the steps of: (a) isolating at least two autologous stem cells from said individual; (b) placing said stem cells in an environment such that the stem cells will mature into cardiac myocytes; (c) imaging said cardiac myocytes using super-resolution fluorescence microscopy to determine the spatial arrangement of the proteome; and (d) comparing the spatial arrangement of the proteome to a physical model, wherein a fissure width of greater than a reference value as derived from the physical model indicates that the individual is likely to develop familial cardiomyopathy, and wherein a fissure width of less than the reference value as derived from the physical model indicates that the individual is unlikely to develop familial cardiomyopathy.

In some embodiments, the cardiac myocytes are human induced pluripotent stem cell-derived. In further embodiments, the individual is between the ages of birth and 18 years old. The imaging method may, in some embodiments, have a resolution of about 20 nm, or, in further embodiments, may comprise direct stochastic optical reconstruction microscopy (dSTORM). The spatial arrangement of the cardiac myocytes may be determined using any relevant technique, such as a proximity ligation assay.

In another aspect, the present invention provides a method of screening a compound or therapeutic agent for efficacy against an individual's distinct molecular phenotype of a genetic disease or abnormality, said method comprising the steps of: (a) obtaining one or more autologous stem cells from said individual; (b) placing said one or more stem cells in an environment such that the stem cells will mature into derived cells expressing the individual's distinct molecular phenotype: (c) imaging said derived cells using super-resolution fluorescence microscopy to determine protein localization; (d) treating said derived cells with the compound or therapeutic agent; (e) imaging the treated derived cells using super-resolution fluorescence microscopy to determine protein localization; and (t) comparing the protein localization of the derived cells with the protein localization of the treated derived cells, wherein a change in localization is statistically significant and the change brings the localization closer to the healthy phenotype indicates efficacy of the compound or therapeutic agent against the individual's distinct molecular phenotype.

In some embodiments, the imaging method may have a resolution of about 20 nm, or, in further embodiments, may comprise direct stochastic optical reconstruction microscopy (dSTORM). In further embodiments, the genetic disease or abnormality may be selected from the group consisting of cardiomyopathy, inherited arrhythmia disorders, cancer, parkinson's disease, alzheimers disease, and autoimmune disease. The molecular target may, in some embodiments, be selected from the group consisting of EGFRs, ERs, molecules that localize to the cardiac intercalated disc, molecules that localize at the cardiac dyad, molecules that conform the cardiac costamere, or structural molecules of the sarcomere.

In yet another aspect, a method is provided for screening a compound or therapeutic agent for efficacy against an individual's distinct molecular phenotype of a cancer characterized by a solid tumor and overexpression of a protein biomarker, said method comprising the steps of: (a) obtaining at least one solid tumor cell; (b) imaging said solid tumor cell at a resolution of about 20 nm to determine the density of the protein biomarker; (d) treating said cell with the compound or therapeutic agent; (e) imaging the treated cell using at a resolution of about 20 nm to determine density of the protein biomarker; and (d) comparing the biomarker density of the cells with the biomarker density of the treated cells, wherein a decrease in the density of the protein biomarker indicates efficacy of the compound or therapeutic agent against the individual's distinct molecular phenotype.

In some embodiments, the imaging method may have a resolution of about 20 nm, or, in further embodiments, may comprise direct stochastic optical reconstruction microscopy (dSTORM). In further embodiments, the protein biomarker is selected from the group consisting of HER2 molecules that localize to the cardiac intercalated disc, molecules that localize at the cardiac dyad, molecules that conform the cardiac costamere, or structural molecules of the sarcomere.

As used herein, “super-resolution” imaging refers to any imaging method, excluding electron microscopy, that achieves a resolution of below 50 nm. Super-resolution imaging methods include, but are not limited to, super-resolution microscopy, super-resolution patch clamp, fluorescent imaging with one nanometer accuracy (FIONA), stochastic optical resolution microscopy (STORM) or direct STORM.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used in this disclosure, except where the context requires otherwise, the term “comprise” and variations of the term, such as “comprising,” “comprises” and “comprised” are not intended to exclude other additives, components, integers or steps.

As used herein, “pluripotent stem cell” means a stem cell that can differentiated into two or more differentiated cell types. For example, differentiated cell types can include blood cells, neural cells, endothelial cells, cardiac cells, pancreatic cells, kidney cells, liver cells, spleen cells and lung cells.

As used herein, the term “isolated” means that materials naturally accompanying a substance in normal circumstances are at least reduced, or substantially or completely eliminated. In some embodiments, an isolated material constitutes at least about 50%, at least about 75%, at least about 80%, at least about 85%, at least about 90%, at least about 95%, or at least about 99% by weight of a sample containing it. The term “isolated cell” refers to a cell substantially free from other accompanying substances present in natural circumstances (e.g., other cells, proteins, nucleic acids, etc.).

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 is a series of micrographs comparing TIRF microscopy and dSTORM. NRVMs stained for Cx43 and PKP2. (A) and (B) show the same region of intercellular contact visualized by TIRF (A) and by dSTORM (B). Small white squares in (A) are enlarged in (C) and (D) to show improved resolution after reconstruction (right). (E) shows a Cx43 cluster surrounded by PKP2, also shown in (F) as a topological image (wherein the z axis shows signal intensity). The dotted line across the image is plotted in (G) to show the intersection of both signals. Scale bars: 5 μm (A and B) and 200 nm (C, D and E);

FIG. 2 shows a Cx43 cluster analysis. (A) shows Cx43 (green) and PKP2 (purple), with an area of overlap (white). Yellow dotted line demarcates area of Cx43 cluster. (B) shows a histogram of area occupied by each Cx43 cluster, with two primary Gaussian peaks centered at 13,313±328 nm2 and 25,035±226\ nm2. (C) shows the cluster circularity index; where a value of 1.0 indicates a perfect circle. (D) shows the correlation between area and perimeter, and (E) shows the correlation between perimeter and circularity; wherein the smaller clusters have a more circular shape. n=136 clusters of 6 images analyzed. Scale bar in (A) 200 nm;

FIG. 3 shows a PKP2 cluster analysis, wherein (A) shows same cluster as FIG. 2A, but in with the yellow dotted line demarcating a PKP2 cluster. (B) shows a frequency distribution of measured PKP2 cluster sizes. Gaussian peaks: 25,094±170 nm2 and 57,421±4,092 nm2. (C) shows the clustercCircularity index; PKP2 clusters are more irregular than Cx43 clusters. (D) shows the linear correlation between area and perimeter, and (E) the correlation between perimeter and circularity indicates that clusters become more irregular when the area and perimeter increase. n=764 clusters of 6 images analyzed. Scale bar in A: 200 nm; and

FIG. 4 is a block diagram of a computer system in accordance with an illustrative implementation.

FIGS. 5A-J. Skp2E3LI, C2, increases nuclear p27 in ECC-1 cells determined by super resolution fluorescent microscopy and quantitation. FIG. 5(A) ECC-1 cells were treated with C2, or veh for 18 hours and prepared as described in Materials and Methods. FIG. 5 (A) One representative ECC-1 cell nucleus treated with veh is outlined (white dashed line) in both a raw total internal reflection fluorescence (TIRF) image (left panel) and super resolved image (right panel). The scale bar corresponds to 3 μm. (FIG. 5B) Selected zoomed regions from the cell treated with veh (panel A) show colocalization between p27 and Skp2 in both TIRF and super resolution formats. The scale bar corresponds to 300 nm. (FIG. 5C) One representative ECC-1 cell nuclei treated with C2 is outlined (white dashed line) in both a raw TIRF image (left panel) and super resolved image (right panel). The scale bar corresponds to 3 μm. (FIG. 5D) Selected zoomed regions from the C2-treated cell in C show colocalization between p27 and Skp2 in both TIRF and super resolution formats. The scale bar corresponds to 300 nm. Both B and D resolve p27 and Skp2 in a complex. (FIG. 5E and FIG. 5H) The protein/cluster density of p27 (FIG. 5E) and Skp2 (FIG. 5H) in ECC-1 cell nuclei treated with veh (n=39 nuclei counted) or C2 (n=40 nuclei counted) is unchanged. Error bars correspond to the SEM. (FIG. 5F and FIG. 5I) C2 significantly increases the area of protein clusters for p27 FIG. 5 (F) compared with veh-treated cells; there is a slight nonstatistically significant increase in Skp2 FIG. 5 (I) compared with veh-treated cells. Error bars correspond to SEM. (FIG. 5G and FIG. 5J) C2 induces an increase in p27 cluster area distribution (size) by associating with existing nuclear p27 FIG. 5 (G) clusters, whereas Skp2 FIG. 5 (J) shows 2 populations; both are compared with veh-treated cells. The data in J shows that after C2-treatment, Skp2 forms a higher-order oligomeric structure with a peak in area at 40 000 nm2.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

The present invention is based upon the discovery that that novel imaging and computational methods can improve accuracy of early-stage diagnostics as well as provide aid in customizing therapeutic regimens. In one aspect, super resolution microscopy is utilized to determine the phenotype, orientation, relative spatial arrangement, or characteristics of molecules or structures within a cell. The phenotype, orientation, relative spatial arrangement, or characteristics may be indicative of a particular disease, trait, or otherwise expressed aspect of an individuals genes. Importantly, these phenotype, orientation, relative spatial arrangement, or characteristics may be present, at the nanometer scale, prior to an observed physical manifestation in an individual.

In one aspect, the invention provides improved methods of early detection and prognosis determination for familial cardiomyopathy. The first manifestation of a cardiomyopathy (before the appearance of the clinical state of the disease) involves changes in the distance between molecules within a proteome. As such, optical tools that allow visualization within the nanometer scale, as well as computational methods to generate true statistical models of fissure development, greatly increase diagnosis capability. In the present application, tools are developed for detection of nanometric separations between molecules within confined subdomains of human cardiac cells. This method is used in the diagnosis of inherited cardiac muscle diseases. The implementation rests on the use of two novel imaging tools: super-resolution microscopy (SRM), and super-resolution patch clamp (SRPC). The implementation also rests on the availability of existing methods by which blood cells from a patient can be transformed in vitro into cardiac cells and as such, studied for their structural composition.

In some cases, inherited arrhythmia disorders do not lead to alterations in the structure of the heart as defined by conventional light microscopy. Yet, molecular separations may be detected at the nanoscale. These diseases include Brugada syndrome, long QT syndrome, and catecholaminergic polymorphic ventricular tachycardia (CPVT).

In many embodiments, the present methods require the harvest of pluripotent stem or progenitor cells, which are then placed in conditions to force differentiation into a desired lineage. As used herein the term “pluripotent stem cells” (PS cells) are cells that are capable under the right conditions of producing progeny of several different cell types. PS cells are capable of producing progeny that are derivatives of each of the three germ layers: endoderm, mesoderm, and ectoderm, according to a standard art-accepted test, such as the ability to form a teratoma in a suitable host, or the ability to differentiate into cells stainable for markers representing tissue types of all three germ layers in culture. Included in the definition of PS cells are embryonic cells of various types, such as embryonic stem (ES) cells, as well as induced pluripotent stem cells (iPS) that have been reprogrammed from an adult somatic cell.

Any known methods are contemplated for use in inducing differentiation of stem cells into cells of the desired lineage, including exposure to any known morphogens. Cell culturing substrates may be made of purified proteins or protein mixtures from a cell line, plant cells, or algae, and may be solid or semi-solid. The substrate may be a hydrogel or other synthetic substrate that may be mixed with proteins, sugars, cytokines, or other molecules that may aid in cell survival and growth. Non-limiting exemplary cell culturing matrices include: proteins secreted by Engelbreth-Holm-Swarm Mouse Tumor cells sold under the trademarks Matrigel™ and Geltrex® LDEV-Free Matrix Products; a defined substrate containing components of human orgin sold under the trademark CellStart™ CTS™; an alginate-based matrix sold under the trademark AlgiMatrix™; a mixture of recombinant human cell adhesion proteins sold under the trademark StemXVivo™ Culture Matrix; a hydrogel peptide scaffold sold under the trademark PuraMatrix™; collagen; laminin; fibronectin; vitronectin; gelatin; and agar.

Any known morphogens may be used to induce differentiation of pluripotent/stem cells into, for example, cardiac myocytes. As used herein, the term “morphogen” refers to a molecule or mixture of molecules that induces differentiation, chemotaxis, and/or proliferation of a PS cell. In one embodiment, the morphogen provides spatial information via a concentration gradient that can affect patterning of a differentiating PS cell culture. In some embodiments, a morphogen is a diffusible protein, cytokine, or growth factor.

Once pluripotent cells have been differentiated as desired, imaging spatial conformation, shape, and dimensions of the cells may be accomplished by any method with a resolution of less than about 50 nm, such as, for example, super-resolution microscopy, super-resolution patch clamp, fluorescent imaging with one nanometer accuracy (FIONA), stochastic optical resolution microscopy (STORM) or digital STORM.

The raw images are then subjected to computational methods comprising the following steps: (i) detection of molecules using an optimized method that reliably can detect molecular features; (ii) building a physical model of the interactions and determining the model parameters using computer simulations and randomly generated images with the same number and density of the proteins of the real image; (iii) applying univariate and multivariate statistical methods to further reduce these model parameters in dimensionality and to obtain a set of biomarkers that can subsequently be combined into multivariate predictive/classifier models, with model error estimated using cross-validation. Any known statistical methods may be used to generate reference values for use in diagnostics, or to generate a standard library for comparison such that disease progression may be tracked.

In another aspect, the present invention provides methods of screening compounds for therapeutic properties against an individual's unique molecular phenotype. For example, certain cancers, while genetically regulated, may express vastly different phenotypes at the molecular level, and, therefore, respond quite differently to the same treatment. Human epidermal growth factor receptor type 2 (HER2, ErbB-2, neu,), as an example, is a well-established tumor biomarker that is overexpressed in a wide variety of carcinomas including breast, ovarian, prostate, and lung cancer. Since HER2 overexpression plays an important role in aggressive tumor behavior and poor clinical outcome, the early-stage detection and quantification of HER2 is clinically relevant and could be used for selection of optimal therapy for individual patients.

There are a number of options for treatment of HER2, each of which may be more or less effective against a given phenotype. For example, HER2 it can be indirectly down-regulated by Hsp90 inhibitors such as the naturally occurring ansamycin antibiotic geldanamycin (GA), or the recently developed 17(dimethylaminoethylamino)-17-demethoxygeldanamycin (17-DMAG), or the original 17-allylamino-17-demethoxygeldanamycin (17-AAG). The earlier the phenotypic response to the possible treatments is known, the better chance of remission.

To screen a compound against an individual's unique molecular phenotype, therefore, the following method is employed: (a) obtaining at least one solid tumor cell; (b) imaging said solid tumor cell at a resolution of about 20 nm to determine the density of the protein biomarker;

(d) treating said cell with the compound or therapeutic agent; (e) imaging the treated cell using at a resolution of about 20 nm to determine density of the protein biomarker; and (d) comparing the biomarker density of the cells with the biomarker density of the treated cells, wherein a decrease in the density of the protein biomarker indicates efficacy of the compound or therapeutic agent against the individual's distinct molecular phenotype.

The invention also contemplates a computer system for use in the diagnostics described herein. FIG. 4 is a block diagram of a computer system in accordance with an illustrative implementation. The computer system or computing device 400 can be used to implement a mobile computing device, cell phones, clients, servers, etc. The computing system 400 includes a bus 405 or other communication component for communicating information and a processor 410 or processing circuit coupled to the bus 405 for processing information. The computing system 500 can also include one or more processors 410 or processing circuits coupled to the bus for processing information. The computing system 400 also includes main memory 415, such as a random access memory (RAM) or other dynamic storage device, coupled to the bus 405 for storing information, and instructions to be executed by the processor 410. Main memory 415 can also be used for storing position information, temporary variables, or other intermediate information during execution of instructions by the processor 410. The computing system 400 may further include a read only memory (ROM) 410 or other static storage device coupled to the bus 405 for storing static information and instructions for the processor 410. A storage device 425, such as a solid state device, magnetic disk or optical disk, is coupled to the bus 405 for persistently storing information and instructions.

The computing system 400 may be coupled via the bus 405 to a display 435. An input device 430, such as a keyboard, may be coupled to the bus 405 for communicating information and command selections to the processor 410. In another implementation, the input device 430 has a touch screen display 435. The input device 430 can include a cursor control, such as a mouse, a trackball, or cursor direction keys, for communicating direction information and command selections to the processor 410 and for controlling cursor movement on the display 435.

According to various implementations, the processes described herein can be implemented by the computing system 400 in response to the processor 410 executing an arrangement of instructions contained in main memory 415. Such instructions can be read into main memory 415 from another computer-readable medium, such as the storage device 425. Execution of the arrangement of instructions contained in main memory 415 causes the computing system 400 to perform the illustrative processes described herein. One or more processors in a multi-processing arrangement may also be employed to execute the instructions contained in main memory 415.

### EXAMPLES

### Example 1

### Characterization of Shape and Dimensions of Plagues in Cardiac Myocytes

To demonstrate the efficacy of the claimed methods in characterizing cardiac myocytes, dSTORM was used to characterize the shape and dimensions of Cx43 and PKP2 plaques in cardiac myocytes.

Neonatal rat ventricular myocytes were dissociated from hearts of 3-4 day old rats, following previously described procedures3. Conditions for cell culture, protein detection by Western blot or immunofluorescence microscopy, and for shRNA-mediated loss of expression of AnkG were as previously used in our laboratory9. Proximity Ligation Assays (PLA; also known as “Duolink”) were adapted from previous publications12 and from the manufacturer's instructions. Super-resolution imaging was done using a custom-built fluorescence microscope (Leica DMI3000) configured for total internal fluorescence and highly inclined excitation modes. Super-resolved images were constructed at 20 nrn/pixel using the QuickPALM ImageJ plugin and a mapping procedure written in IDL (Exelis Visual Information Solutions). Images were composed from superposition of 2,000 individual frames acquired during the on-off (“blinking”) transitions of the fluorophore.

Monolayers of neonatal rat ventricular myocytes (NRVMs) were fixed and prepared as for conventional immunofluorescence analysis. Cx43 and PKP2 were irnrnunolocalized using commercially available antibodies and Alexa fluorophores (A568 and A647 for Cx43 and PKP2, respectively). Photoswitching of fluorophores was captured in 2,000 separate frames, and images then composed using customized software. FIG. I shows an image of the site of intercellular contact obtained by conventional TIRF before (A) and after image reconstruction (B). Small white squares in (A) outline areas that are enlarged in (C) and (D), showing diffused diffraction limited resolution of the TIRF image (left), and improved clarity obtained after reconstruction (right). Panel (E) shows an enlarged dSTORM image, highlighting the physical characteristics consistently observed: A semi-circular Cx43 cluster (in green), a neighboring PKP2 cluster of less defined shape (in purple), and an edge of Cx43 where the two clusters overlap (white). Panel shows a topological image, with signal intensity represented in the z-axis. The values of fluorescence amplitude for each fluorophore along a cross-section of the image (dotted line) are plotted in (G). Notice the Gaussian shape of the Cx43 intensity plot, intersected in the descending branch by the PKP2 intensity curve, creating an area of overlap. Overlap was always seen on the edges of the Cx43 plaque, and not in its center; we did not observe instances where Cx43 surrounded the PKP2 signal. In contrast to the normal distribution of fluorescence intensity across Cx43 clusters, many PKP2 intensity plots were non-Gaussian, displaying long plateaus interrupted by dips, likely reflecting two or more separate clusters that were too close to be discerned.

The 2-dimensional d-STORM images (as in 2A) were used to measure area, perimeter and circularity of clusters. As shown in 2B, the area occupied by individual Cx43 clusters ranged from 8,000 to 88,000 nm2, but the histogram revealed two primary peaks, both best-defined by Gaussian functions: one with an average value of 13,313±328 nm2 (+/−SEM) and the other one, at 25,035±227 nm2. The two peaks were statistically different from one another, though the mean value of the first Gaussian corresponded to near half the mean value of the second Gaussian. Clusters were mostly of circular shape, with a circularity index larger than 0.8 for 65.4% of all clusters examined (2C). As shown in 2D and 2E, there was a close correlation between area and perimeter of the clusters, and between perimeter and circularity, with clusters of smaller dimension being of a more circular shape. Clusters with circularity larger than 0.8 were used to characterize the fluorescence intensity profile across the diameter (see red dotted line in 2A; average plot in 2F). Amplitude was normalized to the maximum observed in each cluster and distances measured relative to the peak of the Gaussian curve. Consistently, the intensity of the Cx43 fluorescence signal increased progressively from the periphery to the center, indicating that Cx43 density is highest in the center of the plaque and progressively less toward the periphery, in agreement with previous freeze-fracture images of gap junction plaques (REF). Results similar to those described in FIG. 2 were found in cells treated with oligonucleotides that do not affect expression of relevant proteins (φshRNA). The physical features of Cx43 clusters contrasted with those of PKP2 plaques, which displayed a much broader distribution of area and circularity (FIG. 3A-E), consistent with the idea that PKP2 plaques may be formed by more than one cluster, in very close apposition to each other.

The data showed that most Cx43 clusters are of two defined dimensions: One of approximately 25,000 nm² and the other one of about half that size. A third group corresponded to clusters of larger sizes, though not larger than ˜90,000 nm2. In the Cx43 life cycle, at least three types of Cx43 aggregates can be found at or near the membrane: a hemichannel (formed by oligomerization of Cx43 within one cell), a gap junction (formed by the docking of two hemichannels), and the so-called “connexosomes,” representing large Cx43-rich membrane vesicles, likely targeted for degradation. It was also shown that loss of AnkG expression decreased junctional conductance

### Example 2

### Computational Analysis of Molecular Interactions

The data harvested from the high-resolution images is extracted and processed. A multi-step process to extract the relevant features from the image is conducted, including the following steps: (i) detection of molecules using an optimized method that reliably can detect molecular features; (ii) building a physical model of the interactions and determining the model parameters using computer simulations and randomly generated images with the same number and density of the proteins of the real image; (iii) applying univariate and multivariate statistical methods to further reduce these model parameters in dimensionality and to obtain a set of biomarkers that can subsequently be combined into multivariate predictive/classifier models, with model error estimated using cross-validation. This process starting with the raw images is automated to allow for robust and reproducible discovery of biomarkers that can be used to select optimal treatment, monitor disease progression, and evaluate details of the effect of drugs. The process is optimized for each different application, but, for cardiac myocytes, the model is a random distribution of Cx43 and PKP2, wherein AnkG silencing caused distribution to become increasingly random.

When applied to plaques in cardiac myocytes, Monte-Carlo simulations were performed to compare the observed distribution of colocalization of Cx43 and PKP2 to the case of particles of the same size, shape and density being randomly placed. Both Cx43 and PKP2 were represented as ellipses with their sizes and shapes randomly sampled from the experimentally observed size and shape distributions. These ellipses were then placed in a rectangular box (30,000 nm×1,000 nm) with their positions and orientation randomly chosen from uniform distributions. Overlaps between ellipses representing the same protein were not allowed, and when overlap occurred new positions and orientations were selected randomly. New ellipses were added until the experimentally observed densities were reached for both Cx43 and PKP2. This process was repeated 1000 times for each condition. The experimental data show a peak for large overlaps when AnkG is present and this peak decreases when AnkG is silenced. In contrast, the simulations show a much smaller peak, indicating that the distribution of Cx43 and PKP2 is ordered in the untreated cell, and that AnkG silencing causes the distribution to become more disordered.

### Example 3

### Inhibitors of SCF-Skp2/Cks1

Super Resolution Microscopy was also utilized to study Inhibitors of SCF-Skp2/Cks1 E3 Ligase Block Estrogen-Induced Growth Stimulation and Degradation of Nuclear p27kip1. In many human cancers, the tumor suppressor, p27kip1 (p2′7), a cyclin-dependent kinase inhibitor critical to cell cycle arrest, undergoes perpetual ubiquitin-mediated proteasomal degradation by the E3 ligase complex SCF-Skp2/Cks1 and/or cytoplasmic mislocalization. Lack of nuclear p27 causes aberrant cell cycle progression, and cytoplasmic p27 mediates cell migration/metastasis. It has previously been shown that mitogenic 17-β-estradiol (E2) induces degradation of p27 by the E3 ligase Skp1-Cullin1-F-Box-S phase kinase-associated protein2/cyclin dependent kinase regulatory sub-unit 1 in primary endometrial epithelial cells and endometrial carcinoma (ECA) cell lines, suggesting a pathogenic mechanism for type I ECA, an E2-induced cancer. The current studies show that treatment of endometrial carcinoma cells-1 (ECC-1) with small molecule inhibitors of Skp2/Cks1 E3 ligase activity (Skp2E3LIs) stabilizes p27 in the nucleus, decreases p27 in the cytoplasm, and prevents E2-induced proliferation and degradation of p27 in endometrial carcinoma cells-1 and primary ECA cells. Furthermore, Skp2E3LIs increase p27 half-life by 6 hours, inhibit cell proliferation (IC50, 14.3M), block retinoblastoma protein (pRB) phosphorylation, induce G1 phase block, and are not cytotoxic. Similarly, using super resolution fluorescence localization microscopy and quantification, Skp2E3LIs increase p27 protein in the nucleus by 1.8-fold. In vivo, injection of Skp2E3LIs significantly increases nuclear p27 and reduces proliferation of endometrial epithelial cells by 42%-62% in ovariectomized E2-primed mice. Skp2E3LIs are specific inhibitors of proteolytic deg-radation that pharmacologically target the binding interaction between the E3 ligase, SCF-Skp2/Cks1, and p27 to stabilize nuclear p27 and prevent cell cycle progression. These targeted inhibitors have the potential to be an important therapeutic advance over general proteasome inhibitors for cancers characterized by SCF-Skp2/Cks1-mediated destruction of nuclear p27. (Endocrinology 154: 4030-4045, 2013).

Method: Direct excitation fluorescence localization microscopy was used to acquire pixel data that were then transformed into superhigh resolution images. ECC-1 cells, at a density of 1.2 105/slide, were seeded on gelatin-coated (2%) glass slides (gelatin type B; Sigma). The cells were grown to 60% confluency, synchronized by serum starvation for 24 hours and treated with the Skp2E3LI, C2 (10 μM) for 18 hours, or veh (0.1% dimethyl sulfoxide). Cells were washed with PBS, treated with cold Ex-traction buffer (10 mM HEPES-KOH [pH 7.4], 300 mM sucrose, 100 mM NaCl, 3 mM MgCl2, and 0.5% Triton X-100) for 2 minutes to remove the cytoplasm, washed with PBS, and fixed with 4% paraformaldehyde for 30 minutes. Nonspecific binding sites were blocked with 2% glycine, 2% BSA, 0.2% gelatin, and 50 mM NH4Cl in PBS for 60 minutes at room temperature, and the slides were incubated overnight at 4° C. with mouse antihu-man p27 (1:300, clone 57; BD Transduction Labs) or rabbit antihuman p45/Skp2 (1:300, clone H-435; Santa Cruz Biotech-nology, Inc). Secondary antibodies coupled to Alexa Fluor 568 for Skp2 and Alexa Fluor 647 for p27 were incubated with the slides for 30 minutes. A custom-built microscopy setup (39-41), based on a Leica DMI3000 microscope equipped with an HCX PL APO 63X NA 1.47 OIL CORR total internal reflection flu-orescence microscopy objective followed by achromatic 2 tube lens magnification, was used; total magnification was 126. The samples were excited at 532 and 645 nm by lasers in highly inclined illumination mode.

Super resolution fluorescence microcopy imaging was used to determine the effect of the Skp2E3LI, C2, on the spatial organization and relation-ship between p27 and Skp2 in situ in the nuclei of ECC-1 cells. Precise molecular shapes and locations relative to cellular ultrastructure can be obtained by SRFLM, small differences in the in situ cellular configurations of these proteins in the nucleus can be observed in response to Skp2E3LI activity (as shown in FIG. 5, A and C, right panels, compared with the raw fluorescence image, left panels). SRFLM (FIG. 5, A and C, right panels) reveals a discrete nuclear organization of p27 (pink) and Skp2 (green) in veh (FIG. 2A) and C2-treated cells (FIG. 2C). Areas of unique (pink or green) and co (white-yellow)-localization of p27 and Skp2 are observed in both the veh-treated (FIG. 5B) and C2-treated cells (FIG. 2D). The density or distribution of p27 protein clusters in the nucleus (clusters per nm2) is not changed by C2 treatment (FIG. 5E). However, the average size of the clusters is significantly increased (1.8-fold increase) (FIG. 5F). This means that C2 treatment of the cells induces an increase in the amount of p27 in the nu-cleus in the form of greater aggregation at specific sites already present in the nucleus rather than the appearance of more numerous protein clusters. Plotting the histogram of cluster sizes shows that larger p27 clusters are rare, and the increase in the average size derives from increases in the largest clusters but not all clusters (FIG. 5G). Along with the data on the activity of C2 and its specificity for p27 degradation inhibition (30), this observation suggests that a threshold p27 cluster size is necessary for G1 arrest and that just a few clusters reaching this threshold size in the nucleus is sufficient to arrest the cell cycle. The density of Skp2 clusters is also not changed by C2 treatment, and their average size is increased but not as significantly (FIGS. 5 H and I). The increase in p27 protein and slight increase in Skp2 in response to C2 observed by Super resolution microscopy is consistent with the amounts of these proteins obtained in FIG. 1C by immunoblotting. Unlike p2′7, however, the histogram of Skp2 clusters is bimodal with a second peak of clusters around 30 000-50 000 nm2 size (FIG. 5J), but this distribution is relatively unaffected by C2 treatment.

This distribution suggests the ubiquitous (rather than cell cycle phase specific) presence of 2 different forms of Skp2 clusters, the second being a higher-order oligomeric structure. Because Skp2 has several substrates, it is possible that only 1 of the 2 detected forms is specific for p27. Super resolution microscopy can be used to resolve subdiffraction features of molecular clusters as shown here, these inhibitors of Skp2 ubiquity-lation of p27 can be used as chemical probes to map the spatial organization and interactions of nuclear cell cycle proteins during Skp2E3LI-induced G1. Future Super resolution microscopy studies may confirm the molecular environment of these Skp2 forms within chromatin, eg, relative to the location of origins of DNA replication. These studies demonstrate the utility of Super resolution microscopy used in conjunction with specific target-based chemical probes, such as Skp2E3LIs.

What is claimed is:

1. A method of determining the prognosis for an individual with a genetic predisposition for a determined phenotype comprising:
(a) isolating at least two autologous stem cells from said individual;
(b) inducing differentiation of said stem cells into differentiated cells;
(c) imaging said differentiated cells to determine a property of the proteome; and
(d) comparing a property of the proteome to a physical model to determine variance from a property reference value.

2. The method of claim 1, wherein the property is spatial arrangement and comparing comprises comparing the spatial arrangement of the proteome to a physical model to determine variance from a spatial arrangement reference value

3. The method of claim 2, wherein the determined variance corresponds to a fissure width and fissure width of greater than a reference value as derived from the physical model indicates that the individual is likely to develop a disease or disorder associated with the phenotype, and wherein a fissure width of less than the reference value as derived from the physical model indicates that the individual is unlikely to develop the disease or disorder.

4. The method of claim 3, wherein the determined phenotype is familial cardiomyopathy, wherein the inducing of differentiation comprises inducing differentiation of said stem cells into cardiac myocytes; and wherein a fissure width of greater than a reference value as derived from the physical model indicates that the individual is likely to develop familial cardiomyopathy, and wherein a fissure width of less than the reference value as derived from the physical model indicates that the individual is unlikely to develop familial cardiomyopathy.

5. The method of claim 4, wherein the cardiac myocytes are human induced pluripotent stem cell-derived.

6. The method of claim 4, wherein the spatial arrangement of the cardiac myocytes is determined using a proximity ligation assay.

7. The method of claim 1, wherein the imaging method is super-resolution fluorescence microscopy.

8. The method of claim 1, wherein the imaging method has a resolution of about 20 nm.

9. The method of claim 1, wherein the imaging method is direct stochastic optical reconstruction microscopy (dSTORM).

10. The method of claim 1, wherein the determined phenotype is an E2-induced cancer, wherein the inducing of differentiation comprises inducing differentiation of said stem cells into endometrial cells; and wherein a fissure width of greater than a reference value as derived from the physical model indicates that the individual is likely to develop from the physical model indicates that the individual is unlikely to develop E2-indeuced cancer.

11. The method of claim 1, wherein the property of the proteome is electric activity.

12. A method of screening a compound or therapeutic agent for efficacy against an individual's distinct molecular phenotype of a genetic disease or abnormality, said method comprising the steps of:
(a) Obtaining one or more autologous stem cells from said individual;
(b) Placing said one or more stem cells in an environment such that the stem cells will mature into derived cells expressing the individual's distinct molecular phenotyupe;
(c) Imaging said derived cells to determine protein localization;
(d) Treating said derived cells with the compound or therapeutic agent;
(e) Imaging the treated derived cells using super-resolution fluorescence microscopy to determine protein localization; and
(f) Comparing the protein localization of the derived cells with the protein localization of the treated derived cells,
wherein the statistically significant change in localization such that the localization is closer to the healthy phenotype indicates efficacy of the comound or therapeutic agent against the individual's distinct molecular phenotype.

13. The method of claim 12, wherein the imaging method is super-resolution fluorescence microscopy.

14. The method of claim 12, wherein the imaging method is direct stochastic optical reconstruction microscopy (dSTORM).

15. The method of claim 12, wherein the molecular target is selected from the group consisting of EGFRs, ERs, molecules that localize to the cardiac intercalated disc, molecules that localize at the cardiac dyad, molecules that conform the cardiac costamere, or structural molecules of the sarcomere.

16. The method of claim 12, wherein the imaging method has a resolution of about 20 nm or greater.

17. A method of screening a compound or therapeutic agent for efficacy against an distinct molecular phenotype of a cell relating to a protein biomarker, said method comprising the steps of:
(a) obtaining at least one cell;
(b) imaging said cell at a resolution of about 20 nm or greater to determine the density of the protein biomarker;
(c) treating said cell with the compound or therapeutic agent;
(d) imaging the treated cell using at a resolution of about 20 nm or greater to determine density of the protein biomarker; and
(e) comparing the biomarker density of the cells with the biomarker density of the treated cells,
wherein a change in the density of the protein biomarker indicates efficacy of the compound or therapeutic agent against the individual's distinct molecular phenotype.

18. The method of claim 17, wherein the cell is a cancer characterized by a solid tumor and overexpression of the protein biomarker, wherein a decrease in the density of the protein biomarker indicates efficacy of the compound or therapeutic agent against the cell's molecular phenotype.

19. The method of claim 17, wherein the protein biomarker is selected from the group consisting of HER2, ER, EGFR, BRCA 1/2, and P53.

20. The method of claim 17, wherein the steps of imaging are carried out using super-resolution fluorescence microscopy.

21. A system comprising:
a processor; and
a tangible computer-readable medium operatively connected to the processor and an image capture mechanism and including computer code configured to:
detect of molecules using an optimized method that reliably can detect molecular features;
build a physical model of the interactions and determining the model parameters using computer simulations and randomly generated images with the same number and density of the proteins of the real image;
apply statistical methods to further reduce these model parameters in dimensionality; and
obtain information regarding a phenotype.
